(19) United States
(12) Patent Application Publication
(10) Pub. No.: US 2025/0270261 A1
(43) Pub. Date: Aug. 28, 2025

(54) AAV CAPSID PROTEINS FOR NUCLEIC ACID TRANFER

(71) Applicant: The Board of Trustees of the Leland Stanford Junior University, Stanford, CA (US)

(72) Inventors: Mark A. Kay, Palo Alto, CA (US); Adriana Verenisse Gonzalez Sandoval, Palo Alto, CA (US)

(21) Appl. No.: 18/858,262

(22) PCT Filed: Apr. 20, 2023
(86) PCT No.: PCT/US2023/066034

§ 371 Date: Oct. 18, 2024

Related U.S. Application Data
(60) Provisional application No. 63/333,459, filed on Apr. 21, 2022.

(51) Int. Cl. C07K 14/005; C12N 15/86

(52) U.S. Cl. CPC C07K 14/005; C12N 15/86; C12N 2750/14122; C12N 2750/14143

(57) ABSTRACT

Recombinant adeno-associated viral (rAAV) vectors expressing recombinant capsid proteins, having enhanced transduction properties in humans and mice are provided. Methods for generating the rAAV capsid proteins and assays to assess transduction efficiency are also provided. Also described are methods for determining the therapeutic efficacy for a gene of interest in two species of mammals by delivering to them the rAAV carrying the gene of interest encapsidated in the recombinant capsid protein.

### STATEMENT REGARDING FEDERALLY SPONSORED RESEARCH

This invention was made with Government support under contract AI116698 awarded by the National Institutes of Health. The Government has certain rights in this invention.

### REFERENCE TO SEQUENCE LISTING, TABLE OR COMPUTER PROGRAM

The instant application contains a Sequence Listing which has been filed electronically in XML format and is hereby incorporated by reference in its entirety. Said XML copy, created on Apr. 19, 2023, is named “091511-0648_SEQ” and is 7,144 bytes in size.

### TECHNICAL FIELD

The subject matter described herein relates to nucleotide sequence encoding recombinant adeno-associated viral (rAAV) capsid protein having enhanced transduction properties across species. The subject matter also relates to plasmids and viruses comprising the identified sequence to provide high transduction efficiency and a low level of neutralization by the human immune system. The subject matter also relates to methods of transferring a nucleic acid sequence of interest by employing rAAV vectors encoding recombinant capsid proteins. The subject matter also relates to methods for determining the therapeutic efficacy for a gene of interest in rodents and humans.

### BACKGROUND

Multiple recombinant gene transfer vectors based on different types of viruses have been developed and tested in clinical trials in recent years. Gene transfer vectors based on adeno-associated virus (AAV), i.e., AAV vectors, have become favored vectors because of characteristics such as an ability to transduce different types of dividing and non-dividing cells of different tissues and the ability to establish stable, long-term transgene expression. While vectors based on other viruses, such as adenoviruses and retroviruses may possess certain desirable characteristics, the use of other vectors has been associated with toxicity or some human diseases. These side effects have not been detected with gene transfer vectors based on AAV (Manno et al., Nature Medicine, 12 (3): 342 (2006)). Additionally, the technology to produce and purify AAV vectors without undue effort has been developed.

At least eleven AAV serotypes have been identified, cloned, sequenced, and converted into vectors, and at least 100 new AAV variants have been isolated from non-primates, primates and humans. However, the majority of preclinical data to date involving AAV vectors has been generated with vectors based on the human AAV-2 serotype, considered the AAV prototype.

There are several disadvantages to the currently used AAV-2 vectors. For example, a number of clinically relevant cell types and tissues are not efficiently transduced with these vectors. Also, a large percentage of the human population is immune to AAV-2 due to prior exposure to wildtype AAV-2 virus. It has been estimated that up to 96% of humans are seropositive for AAV-2, and up to 67% of the seropositive individuals carry neutralizing anti-AAV-2 antibodies which could eliminate or greatly reduce transduction by AAV-2 vectors. Moreover, AAV-2 has been reported to cause a cell mediated immune response in patients when given systemically (Manno et al., Nature Medicine, 12 (3):342 (2006)).

Methods of overcoming the limitations of AAV-2 vectors have been proposed. For example, randomly mutagenizing the nucleotide sequence encoding the AAV-2 capsid by error-prone PCR has been proposed as a method of generating AAV-2 mutants that are able to escape the neutralizing antibodies that affect wildtype AAV-2. However, it is expected that it will be difficult to generate significantly improved AAV-2 variants with single random point mutations, as the naturally occurring serotypes have, at most, only about 85% homology in the capsid nucleotide sequence.

Methods of using a mixture of AAV serotype constructs for AAV vectors have also been developed. The resulting chimeric vectors possess capsid proteins from different serotypes, and ideally, have properties of the different serotypes used. However, the ratio of the different capsid proteins is different from vector to vector and cannot be consistently controlled or reproduced (due to lack of genetic templates), which is unacceptable for clinical use and not satisfactory for experimental use.

The recombinant AAV vector, AAV-LK03, the subject of U.S. Pat. No. 9,169,299, exhibits 30-times better transduction than AAV-DJ (U.S. Pat. No. 8,067,014). AAV-LK03 is primate specific and therefore preclinical studies, typically performed in non-primates, particularly rodents require the use of surrogate serotypes leading to higher costs and time to preclinical development. AAV based vectors that can be used efficiently across species are absent in the art. There is therefore an urgent need for AAV vectors with cross species compatibility, so the need for surrogate serotypes can be circumvented. The present invention addresses this long-standing need in the art.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a recombinant capsid protein, and methods for generating the recombinant capsid protein are provided. The capsid protein includes regions or domains that are derived from different serotypes of AAV. The AAV serotypes may be human or non-human. Recombinant AAV comprising the capsid proteins and plasmids encoding the capsid proteins are also provided.

In one aspect, a capsid protein comprises a first amino acid sequence identified in SEQ ID NO: 1 (AAV-AM).

In one embodiment, the capsid protein comprises a sequence of amino acid residues substantially similar to or substantially identical to AAV-LK03 except for an additional Glycine residue that is inserted into the amino acid sequence in AAV-LK03.

In some aspects, the capsid protein is encoded by a nucleotide sequence identified in SEQ ID NO: 2, or a sequence having at least about 85% sequence identity, at least about 90% sequence identity, at least about 95% sequence identity, at least about 96% sequence identity, at least about 97% sequence identity, at least about 98% sequence identity, or at least about 99% sequence identity thereto.

In one embodiment, the capsid protein comprises an amino acid sequence shown in SEQ ID NO: 1 that is encoded by the nucleotide sequence identified by SEQ ID NO: 2.

In one embodiment, the capsid protein has a sequence that has at least about 90%, 91%, 92%, 93%, 94%, 95%, 96%, 97%, 98%, or 99% to SEQ ID NO: 1, with the proviso that the protein is not identical to SEQ ID NO: 3. In one embodiment, the capsid protein is not SEQ ID NO: 3.

A viral particle comprising a capsid protein sequence as described above, is contemplated in some embodiments. Disclosed herein are viral particles comprising the capsid proteins encoded by the nucleotide sequences identified by SEQ ID NO: 2 of the sequence listing, or a nucleotide sequence having at least 95% sequence identity to said sequence.

Also disclosed is a plasmid comprising the nucleotide sequence identified by SEQ ID NO: 2, or a sequence having at least 95% sequence identity thereto.

Also disclosed is a recombinant AAV vector (rAAV), comprising a capsid protein having an amino acid sequence encoded by a nucleotide sequence identified by SEQ ID NO: 2, or a nucleotide sequence having at least 95% sequence identity thereto.

The present disclosure also provides a method of transfer of a nucleic acid sequence of interest into a cell or into mammal, comprising introducing a recombinant AAV (rAAV) vector into a cell or a mammal, the recombinant AAV vector encoding a gene of interest which is encapsidated into a recombinant capsid protein identified by an amino acid sequence of SEQ ID NO: 1 (AAV-AM).

The present disclosure further provides a method of transferring a nucleic acid of interest into a cell or into mammal, comprising introducing a recombinant AAV (rAAV) vector into a cell or a mammal, the recombinant AAV vector encoding a recombinant capsid nucleotide sequence and a gene of interest, wherein the gene of interest is encapsidated into a recombinant capsid protein having an amino acid encoded by a nucleotide sequence identified in SEQ ID NO: 2, or a sequence having at least about 85% sequence identity, at least about 90% sequence identity, at least about 95% sequence identity, at least about 96% sequence identity, at least about 97% sequence identity, at least about 98% sequence identity, or at least about 99% sequence identity thereto. In this embodiment the rAAV vector is generated by steps comprising, performing a site directed mutagenesis (SDM) of an AAV-LK03 plasmid (U.S. Pat. No. 9,169,299) using overlapping primers comprising an extraneous triplet nucleotide sequence, performing an exponential amplification by PCR using the AAV-LK03 plasmid as template and primer pairs comprising the extraneous triplet nucleotide sequence to generate amplicons, incubating the amplicons with a kinase, Ligase and DpnII, performing a high efficiency transformation in bacteria, culturing the bacteria in the presence of a selection antibiotic to isolate bacterial clones comprising the nucleic acid sequence of interest, confirming by sequencing the presence of the recombinant capsid nucleotide sequence, and purifying plasmids comprising the nucleic acid sequence of interest from the bacterial clones to generate the rAAV vector.

In yet another embodiment, a method of determining the therapeutic efficacy for a gene of interest is provided comprising, delivering to a first mammal, a recombinant AAV (rAAV) vector comprising a nucleotide sequence encoding a recombinant capsid protein with an amino acid sequence as set forth in SEQ ID NO: 1 (AAV-AM), and a gene of interest, wherein the gene of interest is encapsidated into the recombinant capsid protein, assessing an efficiency of transduction in the first mammal, delivering to a second mammal, the rAAV vector, when the efficiency of transduction in the first mammal is higher than that observed after infecting a first mammal with a control rAAV vector, assessing an efficiency of transduction in the second mammal, whereby, a higher efficiency of transduction of the rAAV vector in the first mammal and the second mammal over the control rAAV vector is indicative of a higher therapeutic efficacy for the gene of interest.

Adeno-associated has a broad host range, i.e., it can infect many mammalian cell lines or primary cells, including replicative as well as non-replicative cells, and including cells resident in tissues of the mammal. Some lymphoid cell lines may be more resistant to Adeno-associated virus infection, and thus may need high quantities of viruses to achieve sufficient infection levels. Cell types that may be used in the methods disclosed herein include, but are not limited to, CHO cells, monocytes, dendritic cells (DCs), freshly isolated human blood myeloid DCs, plasmacytoid DCs and monocyte-derived DCs, Langerhans cells and dermal DCs, Human T cell leukemia DND-41 cells, p53-deficient cancer cells, tumor cells retaining wild-type p53, tumor cells of unknown p53 status, adenocarcinoma human alveolar basal epithelial cells, also known as “A549 cells,” human KB cells, Madin Darby Bovine Kidney (MDBK) cells, Mouse Hepa1-6 cells, Mouse Embryonic Fibroblasts (MEF cells), human HuH-7 cells, pulmonary artery endothelial cells (hPAEC), NIH-3T3 cells, Hep G2 cells, HEp-2 cells, HeLa cells, Dempsey cells, human embryonic kidney 293 cells (also known as “HEK 293” or “293 cells”), fetal rhesus monkey kidney (FRhK-4) cells, rat hepatoma H4TG cells, LMH chicken hepatoma epithelial cells, primary human hepatocytes and primary human keratinocytes. In some aspects, the rAAV is used to infect 293 cells. In some aspects, the rAAV is used to infect Hepa1-6 cells. In some aspects, the rAAV is used to infect HuH-7 cells. In some embodiments a helper Adenovirus is used.

In some embodiments, humanized FRG mice are transfected with an AAV in vitro selected library. In some embodiments, non-humanized FRG mice are transfected with an AAV in vitro selected library.

In addition to the exemplary aspects and embodiments described above, further aspects and embodiments will become apparent by reference to the drawings and by study of the following descriptions.

### BRIEF DESCRIPTION OF THE FIGURES

FIGS. 1A-1F show transduction efficiency of AAV carrying the AAV-LK03 (also referred to as LK03) or AAV-AM vectors (14 vg/cell) in human (HuH-7) and mouse (Hepa1-6) cells. FIG. 1A shows luciferase activity 16 h following transduction with LK03 or AAV-AM. FIG. 1B shows luciferase activity 48 h following transduction with LK03 or AAV-AM. FIG. 1C shows assessment of luciferase mRNA expression by qPCR 16 h following transduction with LK03 or AAV-AM. FIG. 1D shows assessment of luciferase mRNA expression by qPCR 48 h following transduction with LK03 or AAV-AM. FIG. 1E shows assessment of Nuclear Vector Copy Number by qPCR 16 h after transduction with LK03 or AAV-AM. FIG. 1F shows assessment of Nuclear Vector Copy Number by qPCR 48 h after transduction with LK03 or AAV-AM.

FIGS. 2A-2B shows the results of in vivo luciferase reporter assays showing transduction efficiency in mice following intravenous injection. FIG. 2A shows luciferase activity in animals on day 3 post injection of AAV-LK03 (3e11 viral genome) Exposure time 120 sec. FIG. 2B shows luciferase activity in animals on day 3 post injection of AAV AAV-AM (3e11 viral genome) Exposure time 1 sec.

FIGS. 3A-3F show assessment of mouse tissue for transduction efficiency of AAV carrying the LK03 or AAV-AM vectors following intravenous injection. FIG. 3A shows luciferase activity 3 days following transducing animals with LK03 or AAV-AM (3e11 viral genome). FIG. 3B shows luciferase activity 15 days following transducing animals with LK03 or AAV-AM. FIG. 3C shows assessment of luciferase mRNA expression by qPCR 3 days following transducing animals with LK03 or AAV-AM. FIG. 3D shows assessment of luciferase mRNA expression by qPCR 15 days following transducing animals with LK03 or AAV-AM. FIG. 3E shows assessment of Nuclear Vector Copy Number by qPCR 3 days after transducing animals with LK03 or AAV-AM. FIG. 3F shows assessment of Nuclear Vector Copy Number by qPCR 15 days after transducing animals with LK03 or AAV-AM.

FIG. 4 is a predictive model for AAV-AM in comparison to AAV-LK03.

### BRIEF DESCRIPTION OF THE SEQUENCES

SEQ ID NO: 1 is an amino acid sequence encoded by SEQ ID NO: 2 and referred to herein as AAV-AM. See Table 1.

It is to be understood that each of the nucleotide sequences disclosed herein can be translated to predict an amino acid sequence representing a rAAV capsid protein.

### DETAILED DESCRIPTION

Several embodiments of the present disclosure are described in detail hereinafter. These embodiments may take many different forms and should not be construed as limited to those embodiments explicitly set forth herein. Rather, these embodiments are provided so that this disclosure will be thorough and complete, and will fully convey the scope of the present disclosure to those skilled in the art.

### I. DEFINITIONS

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described.

It must be noted that as used herein and in the appended claims, the singular forms “a”, “an”, and “the” include plural referents unless the context clearly dictates otherwise. Thus, for example, reference to “a protein” includes more than one protein, and reference to “a compound” includes more than one compound. In describing and claiming the present invention, the following terminology will be used in accordance with the definitions set out below:

A polynucleotide is typically composed of a specific sequence of four nucleotide bases: adenine (A); cytosine (C); guanine (G); and thymine (T) (uracil (U) for thymine (T) when the polynucleotide is RNA). Unless specifically delineated, the abbreviated nucleotides may be either ribonucleosides or 2′-deoxyribonucleosides. The nucleosides may be specified as being either ribonucleosides or 2′-deoxyribonucleosides on an individual basis or on an aggregate basis. When specified on an individual basis, the one-letter abbreviation is preceded by either a “d” or an “r,” where “d” indicates the nucleoside is a 2′-deoxyribonucleoside and “r” indicates the nucleoside is a ribonucleoside. For example, “dA” designates 2′-deoxyriboadenosine and “TA” designates riboadenosine. When specified on an aggregate basis, the particular nucleic acid or polynucleotide is identified as being either an RNA molecule or a DNA molecule. Nucleotides are abbreviated by adding a “p” to represent each phosphate, as well as whether the phosphates are attached to the 3′-position or the 5′-position of the sugar. Thus, 5′-nucleotides are abbreviated as “pN” and 3″-nucleotides are abbreviated as “Np.” where “N” represents A, G, C. T or U. When nucleic acid sequences are presented as a string of one-letter abbreviations, the sequences are presented in the 5′->3′ direction in accordance with common convention, and the phosphates are not indicated. Thus, the term polynucleotide sequence is the alphabetical representation of a polynucleotide molecule. This alphabetical representation can be input into databases in a computer having a central processing unit and used for bioinformatics applications such as functional genomics and homology searching.

An “isolated polynucleotide” molecule is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences in association therewith.

Techniques for determining nucleic acid and amino acid “sequence identity” also are known in the art. Typically, such techniques include determining the nucleotide sequence of the mRNA for a gene and/or determining the amino acid sequence encoded thereby and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul. Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the proteins being compared. Default parameters are provided to optimize searches with short query sequences in, for example, blastp with the program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

Alternatively, the degree of sequence similarity between polynucleotides can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80-85%, preferably 85-90%, more preferably 90-95%, and most preferably 98-100% sequence identity to the reference sequence over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art.

### II. CHIMERIC AAV CAPSIDS

Capsid proteins may be derived from two or more different serotypes of AAV. For example, a capsid protein can have a first region that is derived from or having high levels of sequence similarity or identity to a first AAV serotype or known recombinant AAV capsid protein (e.g., AAV-DJ), a second region similarly derived from or having high levels of sequence similarity or identity to a second AAV serotype or known recombinant AAV capsid protein, as well as third, fourth, fifth, six, seventh and eighth regions, etc. derived from or having high levels of sequence similarity or identity to another AAV serotype or known recombinant AAV capsid protein. The AAV serotypes may be human AAV serotypes or non-human AAV serotypes, such as murine, bovine, avian, and caprine AAV serotypes. In particular, non-primate mammalian AAV serotypes, such as AAV sequences from rodents (e.g., mice, rats, rabbits, and hamsters) and carnivores (e.g., dogs, cats, and raccoons), may be used. By including individual amino acids or regions from multiple AAV serotypes in one capsid protein, capsid proteins that have multiple desired properties that are separately derived from the multiple AAV serotypes may be obtained.

The capsid protein referred to herein as “AAV-LK03” is mostly derived from AAV3B with the exception of a hyper-variable region in the 5″ of the gene and a single point mutation at the 3′ end of the gene. It was derived through an AAV-shuffled library screen selective for primate cells and found to transduce human cells 30-times better than AAV-DJ. The selectivity was independent of the transgene (gene of interest) or the cell type. The AAV-LK03 capsid protein is described in U.S. Pat. No. 9,169,299 and incorporated by reference herein, in its entirety.

While the embodiments described above are primarily with respect to an rAAV capsid protein, it is recognized that capsids having amino acid and/or nucleotide sequences that are similar in sequence and having the same function may be used and are contemplated. In one embodiment, a recombinant capsid protein having at least about 60% sequence identity, further at least about 70% sequence identity, at least about 75% sequence identity, at least about 80% sequence identity, at least about 85% sequence identity, at least about 90% sequence identity, at least about 95% identity, at least about 96% sequence identity, at least about 97% sequence identity, at least about 98% sequence identity, or at least about 99% sequence identity to the amino acid sequences identified in the sequence listing is contemplated.

It will be appreciated that conservative amino acid substitutions may be in the polypeptide sequence, to achieve proteins having, for example, 60%, 70%, 75% 80%, 85%, 90%, 95%, 96%, 97%, 98% or 99% sequence identity to a polypeptide encoded by a nucleotide sequence disclosed herein, and preferably with retention of activity of the native sequence. Conservative amino acid substitutions, as known in the art and as referred to herein, involve substituting amino acids in a protein with amino acids having similar side chains in terms of, for example, structure, size and/or chemical properties. For example, the amino acids within each of the following groups may be interchanged with other amino acids in the same group: amino acids having aliphatic side chains, including glycine, alanine, valine, leucine and isoleucine; amino acids having non-aromatic, hydroxyl-containing side chains, such as serine and threonine; amino acids having acidic side chains, such as aspartic acid and glutamic acid; amino acids having amide side chains, including glutamine and asparagine; basic amino acids, including lysine, arginine and histidine; amino acids having aromatic ring side chains, including phenylalanine, tyrosine and tryptophan; and amino acids having sulfur-containing side chains, including cysteine and methionine. Additionally, amino acids having acidic side chains, such as aspartic acid and glutamic acid, are considered interchangeable herein with amino acids having amide side chains, such as asparagine and glutamine.

A mouse model system that is severely immunodeficient has been developed. These fumarylacetoacetate hydrolase (Fah)-deficient mice can be pretreated with a urokinase-expressing adenovirus, and then highly engrafted (up to 90%) with human hepatocytes from multiple sources, including liver biopsies. Furthermore, human cells can be serially transplanted from primary donors and repopulate the liver for at least four sequential rounds. The expanded cells displayed typical human drug metabolism. This system provides a robust platform to produce high-quality human hepatocytes for tissue culture. It may also be useful for testing the toxicity of drug metabolites and for evaluating pathogens dependent on human liver cells for replication. (Azuma, et al., (2007) Nature Biotech. 25:903-910).

A humanized mouse model, known as FRG mice (Yecuris Corporation, Portland, OR), has been designed to allow researchers to grow and expand populations of human hepatocytes in vivo for research and drug testing. The FRG model has the genes Fah, Rag, and Ilrg knocked out. Knocking out Fah yields mouse liver damage; the lack of Rag removes the part of the innate immune system that rejects other mouse cells and knocking out Ilrg inactivates the part of the immune system that would prevent engraftment of cells from other species including humans. Thus, the FRG mouse can either be repopulated with human donor cells of choice or repopulated from a pool of prequalified donors. Animals can be provided with human hepatocytes that range from 5-95% of the total liver mass. Non-repopulated FRG mice are also available for use as study controls. In one embodiment, the recombinant AAV capsid protein comprises sequence of amino acid residues derived from a AAV serotype. In one aspect, the first recombinant AAV capsid protein (first capsid protein) has a first amino acid sequence closely homologous to a sequence of amino acids in second capsid protein sequence in the AAV-LK03 serotype. In one embodiment, close homology intends at least about 80% sequence identity. In one embodiment, close homology intends at least about 90% sequence identity. A contiguous sequence of amino acids in such a conserved set may be anywhere from 2 to 500, 2 to 400, 2 to 300, 2 to 200, 2 to 100, or 2 to 50 amino acid residues in length. In one aspect, first recombinant AAV capsid protein has the first amino acid sequence as show in SEQ ID NO: 1, which is identical to the second capsid protein sequence in the AAV-LK03 serotype (SEQ ID NO: 3) except for a single extraneous amino acid residue that is inserted into the second capsid sequence. In one aspect the single extraneous amino acid residue is inserted at position 266 of the second capsid protein sequence. In one aspect of this embodiment, the extraneous amino acid residue is Glycine.

Also in this embodiment, there is provided a viral particle comprising an amino acid sequence for a first recombinant capsid protein. In one aspect, the first recombinant capsid protein has an amino acid sequence as show in SEQ ID NO: 1.

Further in this embodiment, there is provided a plasmid comprising an amino acid sequence for a recombinant capsid protein. In one aspect, the recombinant capsid protein has an amino acid sequence as show in SEQ ID NO: 1.

Also provided is a recombinant AAV vector, comprising a capsid protein with an amino acid sequence shown in SEQ ID NO: 1.

In another embodiment of the present invention, there is provided a first nucleotide sequence encoding a first recombinant capsid protein. In one aspect, the first nucleotide sequence is closely homologous to a nucleotide sequence encoding for a capsid protein in the AAV-LK03 serotype. In one embodiment, close homology intends at least about 85% sequence identity, at least about 90% sequence identity, at least about 95% sequence identity, at least about 96% sequence identity, at least about 97% sequence identity, at least about 98% sequence identity, or at least about 99% sequence identity. In one aspect, the first nucleotide sequence is as show in SEQ ID NO: 2, which is identical to the nucleotide sequence in AAV-LK03 except for an extraneous triplet nucleotide sequence (triplet codon sequence) that is inserted into the nucleotide sequence in AAV-LK03. In one aspect the triplet codon sequence codes of a single extraneous amino acid residue which is inserted at position 266 in the first recombinant AAV capsid protein sequence. In one aspect of this embodiment, the extraneous amino acid residue is Glycine.

Also in this embodiment, there is provided a viral particle comprising an amino acid sequence for a first recombinant capsid protein encoded by the first nucleotide sequence. In one aspect, the first nucleotide sequence amino acid sequence is shown in SEQ ID NO: 2.

Further in this embodiment, there is provided a plasmid comprising an amino acid sequence for a first recombinant capsid protein encoded by the first nucleotide sequence. In one aspect, the first nucleotide sequence is shown in SEQ ID NO: 2.

Also provided is a recombinant AAV vector, comprising a capsid protein that is encoded by the first nucleotide sequence show in SEQ ID NO: 2.

In another embodiment of this invention is a method of transferring a nucleic acid sequence of interest into a cell or into a mammal, comprising introducing a recombinant AAV (rAAV) vector into a cell or a mammal, the rAAV vector encoding a recombinant capsid nucleotide sequence and a gene of interest, wherein the gene of interest is encapsidated into the recombinant capsid protein In one aspect, the recombinant AAV capsid protein has a first amino acid sequence closely homologous to a sequence of amino acids in a second capsid protein sequence in the AAV-LK03 serotype. In one embodiment, close homology intends at least about 80% sequence identity. In one embodiment, close homology intends at least about 90% sequence identity. A contiguous sequence of amino acids in such a conserved set may be anywhere from 2 to 500, 2 to 400, 2 to 300, 2 to 200, 2 to 100, or 2 to 50 amino acid residues in length. In one aspect, first recombinant AAV capsid protein has the first amino acid sequence as show in SEQ ID NO: 1, which is identical to the second capsid protein sequence in the AAV-LK03 serotype (SEQ ID NO: 3) except for a single additional or extraneous amino acid residue that is inserted into the second capsid sequence. In one aspect the single additional or extraneous amino acid residue is inserted at position 266 of the second capsid protein sequence. In one aspect of this embodiment, the extraneous or additional amino acid residue is Glycine.

In another embodiment a method of transferring a nucleic acid sequence of interest into cells or into a mammals comprises introducing a recombinant AAV (rAAV) vector into a cell or a mammal, the recombinant AAV vector encoding a recombinant capsid nucleotide sequence and a gene of interest, wherein the gene of interest is encapsidated into a recombinant capsid protein having an amino acid encoded by a nucleotide sequence encoding a first recombinant capsid protein. In one aspect, the first nucleotide sequence is closely homologous to a nucleotide sequence encoding for a capsid protein in the AAV-LK03 serotype. In one embodiment, close homology intends at least about 85% sequence identity, at least about 90% sequence identity, at least about 95% sequence identity, at least about 96% sequence identity, at least about 97% sequence identity, at least about 98% sequence identity, or at least about 99% sequence identity. In one aspect, the first nucleotide sequence is as show in SEQ ID NO: 2, which is identical to the nucleotide sequence in AAV-LK03 except for an extraneous triplet nucleotide sequence (triplet codon sequence) that is inserted into the nucleotide sequence in AAV-LK03. In one aspect the triplet codon sequence codes of a single extraneous amino acid residue which is inserted at position 266 in the first recombinant AAV capsid protein shown in SEQ ID NO: 1.

In this embodiment, cell types in which the rAAV may be introduced include but are not limited to cell lines and primary cells. Examples of such cells include, CHO cells, monocytes, dendritic cells (DCs), myeloid DCs, plasmacytoid DCs and monocyte-derived DCs, Langerhans cells and dermal DCs, T cell leukemia cells, tumor cells alveolar basal epithelial cells, Madin Darby Bovine Kidney (MDBK) cells, Mouse Hepa1-6 cells, Mouse Embryonic Fibroblasts (MEF), human HuH-7 cells, pulmonary artery endothelial cells (hPAEC), NIH-3T3 cells, Hep G2 cells, HEp-2 cells. HeLa cells, Dempsey cells, human embryonic kidney 293 cells (HEK 293), fetal rhesus monkey kidney (FRhK-4) cells, rat hepatoma H4TG cells, LMH chicken hepatoma epithelial cells, primary human hepatocytes and primary human keratinocytes.

Also in this embodiment, the rAAV may be introduced into mammals including, but not limited to a primate, a non-human primate and rodent (e.g., mice, rats, rabbits, hamsters) and carnivores (e.g., dogs, cats, raccoons). In one aspect, the mammal is a rodent (e.g. mouse), a human, or both a rodent and a human, which is relevant to preclinical testing of the rAAV in the rodent prior to advancement into clinical studies.

Also in this embodiment, there may be a desire to introduce the rAAV to specific mammalian tissues for purposes of therapy. Mammalian tissues include, but are not limited to liver, pancreas, blood, bone, brain, prostate, ovaries, breast, bladder, and muscle. For example, in one aspect, the liver is the desired target of rAAV transduction for gene therapy.

Also provided is a method of generating a recombinant AAV vector having enhanced transduction properties across animal species. The method comprises performing a site directed mutagenesis (SDM) of an AAV-LK03 plasmid, using overlapping primers comprising an extraneous triplet nucleotide sequence. The SDM product is subject to a PCR amplification reaction using the AAV-LK03 plasmid as template and primer pairs comprising the extraneous triplet nucleotide sequence to generate amplicons. The amplicons are incubated with a kinase, Ligase and DpnII, followed by performing a high efficiency transformation in bacteria. The transformed bacteria are cultured using suitable antibiotics for selection pressure to obtain clones that comprise a recombinant nucleic acid sequence of interest. The nucleic acid sequence is verified by sequencing. Plasmids comprising the recombinant capsid nucleic acid sequence are isolated thereby generating the recombinant AAV vector. In one aspect, the recombinant capsid nucleic acid sequence is shown in SEQ ID NO: 2, which encodes a recombinant capsid protein sequence shown in SEQ ID NO: 1.

In another embodiment, there is provided a method of determining the therapeutic efficacy for a gene of interest comprising delivering to a first mammal, a recombinant AAV (rAAV) vector comprising a nucleotide sequence encoding a recombinant capsid protein with an amino acid sequence as set forth in SEQ ID NO: 1 (AAV-AM), and a gene of interest, wherein the gene of interest is encapsidated into the recombinant capsid protein; assessing an efficiency of transduction in the first mammal; delivering to a second mammal, the rAAV vector, when the efficiency of transduction in the first mammal is higher than that observed after infecting a first mammal with a control rAAV vector; assessing an efficiency of transduction in the second mammal, whereby, a higher efficiency of transduction of the rAAV vector in the first mammal and the second mammal over the control rAAV vector is indicative of a higher therapeutic efficacy for the gene of interest.

In this embodiment, in one aspect, the first mammal species is a rodent (e.g., mice, rats, rabbits, hamsters) and the second mammal species is a primate (e.g., human).

In this embodiment, the efficiency of transduction is determined by measuring at least one of, a nuclear vector copy number, expression of the gene of interest, and activity of a protein encoded by the gene of interest.

It will also be appreciated that the recombinant vectors described herein are contemplated for use in methods of expressing a gene of interest in a variety of cells and in mammals. Transduction into cells lines in addition to the cell lines described herein are exemplary, and other cells lines, particularly stem cells, are contemplated. In terms of in vivo use, the method preferably comprises introducing a recombinant AAV (rAAV) into a mammal, the recombinant AAV vector encoding the gene of interest and comprising a first capsid protein with an amino acid sequence having a single extraneous amino acid residue that is inserted into the second capsid sequence. The vector expressing a gene of interest is introduced to the mammal, typically by injection, intravenously, subcutaneously, intraperitoneal, or the like. The gene of interest can be any gene, and many suitable genes for expression for therapeutic or non-therapeutic purposes are readily identified by a skilled artisan. The nucleotide sequence of the gene of interest is typically “operably linked” to one or more other nucleotide sequences, including but not limited to the gene for a selected capsid protein, a promoter, and enhancer, and the like.

In this embodiment, the rAAV may be introduced into any mammal including, but not limited to a primate, a non-human primate, rodent (e.g., mice, rats, rabbits, hamsters), and carnivores (e.g., dogs, cats, raccoons).

A gene is “operably linked” to another nucleotide sequence when it is placed in a functional relationship with another nucleotide sequence. For example, if a coding sequence is operably linked to a promoter sequence, this generally means that the promoter may promote transcription of the coding sequence. Operably linked means that the DNA sequences being linked are typically contiguous and, where necessary to join two protein coding regions, contiguous and in reading frame. However, since enhancers may function when separated from the promoter by several kilobases and intronic sequences may be of variable length, some nucleotide sequences may be operably linked but not contiguous. Additionally, as defined herein, a nucleotide sequence is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide.

### III. EXAMPLES

The following examples are illustrative in nature and are in no way intended to be limiting. For technical procedures, reference can be made to U.S. Pat. No. 8,067,014, which is incorporated by reference herein, in its entirety, as well as to Grimm, D. et al., (Blood, 102:2412-2419 (2003)). For in vivo procedures, and methods of AAV capsid library generation, including generation of the rAAV, AAV-LK03, reference can be made to U.S. Pat. No. 9,169,299, which is incorporated by reference herein, in its entirety.

### Example 1

### Methods for Assessing Transduction Efficiency

### Luciferase Assay

Cells were seeded in tissue culture dishes. After 12-24 h the cells were transduced with rAAV and incubated for an additional 16 h or 48 h to allow expression of luciferase. Luciferase activity was quantitated using ONE-GLOW™ Luciferase assay reagent (Promega). The growth medium was removed, and a 1:4 dilution of the assay reagent in PBS added to the cells. The cells were incubated for 10 min with shaking followed by measurement of luminescence using a luminometer. A plot of Relative Light Unit (RLU) was used for quantification.

Luciferase mRNA Expression by qPCR

Cells were seeded in tissue culture dishes. After 12-24 h the cells were transduced with rAAV and incubated for an additional 16 h or 48 h. Cells were seeded in tissue culture dishes. After 12-24 h the cells were transduced with rAAV and incubated for an additional 16 h or 48 h. The growth medium was removed and TRIZOL™ reagent was added to the cells followed by incubation for 10 min. The samples were then collected, and RNA isolated using Direct-zol RNA miniprep kit (Zymo Research) according to manufacturer's protocol.

50 ng of the isolated RNA was reverse transcribed using High-Capacity RNA-TO-CDNA™ Kit (Applied Biosciences), according to company's protocol.

SYBER Green qPCR with primers for gene of interest (e.g., Luciferase) and a normalization gene (actin) were used in this assay. A plot of relative enrichment to actin was used for quantification.

Nuclear Vector Copy Number by qPCR

Cells were seeded in tissue culture dishes. After 12-24 h the cells were transduced with rAAV and incubated for an additional 16 h or 48 h. The cells were trypsinized, nuclei isolated according to NE-PER Nuclear and Cytoplasmic Extraction Reagents (ThermoFisher Scientific), according to manufacturer's protocol. After washing the nuclear pellet, DNA was extracted using QIAamp DNA extraction kit (Qiagen). SYBER Green qPCR with primers for gene of interest (e.g., Luciferase) and a normalization gene (PTGER2) were used in this assay. A standard curve was used for quantification and the ratio of viral genome (vg) per cell determined and plotted.

### Example 2

### In Vitro Studies

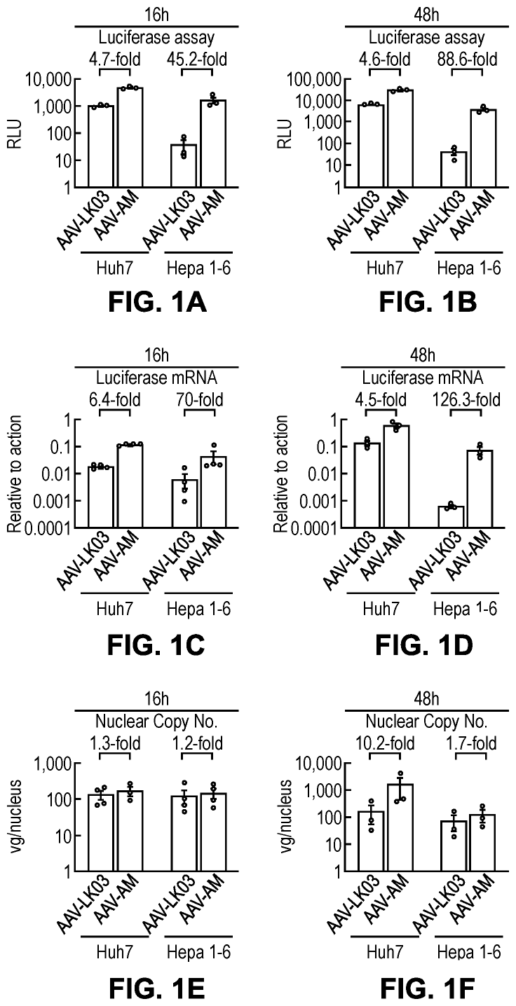

Human (HuH-7) and mouse (Hepa1-6) cells were transduced with control AAV-LK03 or AAV-AM (MOI 1e4) carrying the luciferase reporter gene (surrogate “gene of interest”). Activity of the luciferase protein was quantified using a luminometer as described above. Unexpectedly, transduction with AAV-AM for 16 h resulted in a 4.7-fold higher expression of luciferase in the human cells and 45.2-fold expression of luciferase in mouse cells when compared with AAV-LK03 (FIG. 1A). Expression of luciferase in AAV-AM transduced mouse cells further increased to 88.6-fold after 48 h (FIG. 1B). A similar trend in luciferase mRNA levels was also observed (FIGS. 1C-1D).

Next, the copy number of the vector in the nucleus was determined by qPCR performed after isolating the nuclear fraction from these cells using the method described above. Surprisingly, less that 2-fold difference in nuclear vector copy number was observed between species for AAV-LK03 and AAV-AM (FIGS. 1E and 1F), which could not explain the large differences in mRNA and protein expression (FIGS. 1A-1D).

To further understand this apparent anomaly enrichment of histones and histone modifications associated with the viral genome were investigated. Three core histones, H3, H2A and H4 and four post-translational modifications (PTM) of H3 were analyzed. Of these, H3K4me2 and H3K27ac are generally associated with actively transcribed chromatin, while H3K9me3 and H3K27me3 are repressive histone marks. It was determined that histone H3 and its active PTM, H3K7me3 and H3K27ac were depleted mouse cells transduced with AAV-LK03. In contrast, H3 and its active PTM, H3K7me3 and H3K27ac were enriched in mouse cells transduced with AAV-AM.

These results lead to the hypothesis that the capsid proteins that package or uncoat the viral genome in the nucleus interact with host proteins to help set up the epigenetic state of the viral episome and that these interactions differ between mouse and human species.

### In Vivo Studies

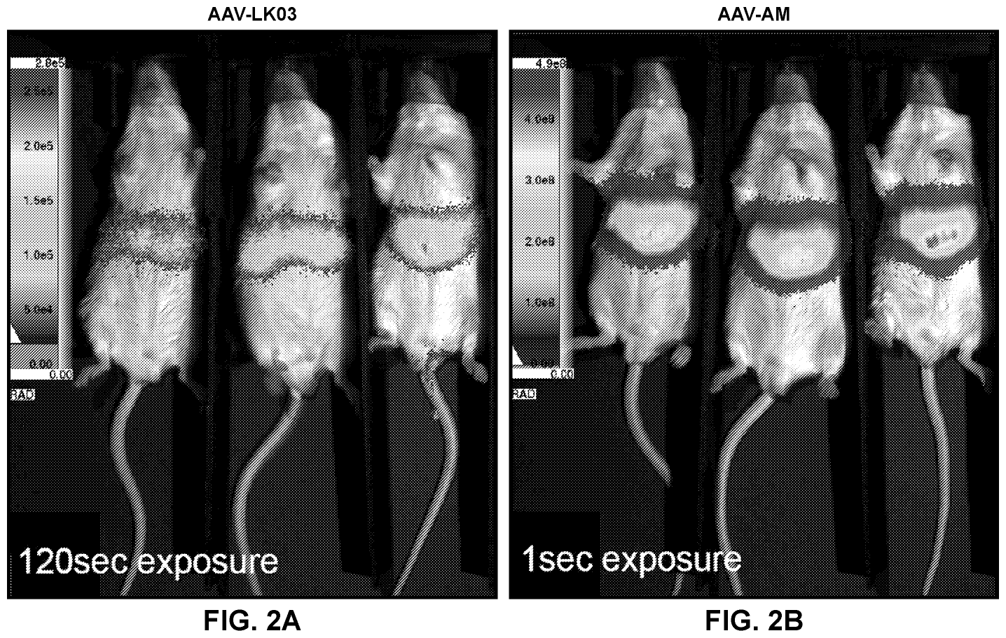

To determine whether the enhanced transduction efficiency with AAV-AM observed in vitro could be reproduced in vivo, BALB/c scid mice injected with control AAV-LK03 or AAV-AM (3e11 viral genome) were imaged after 3 days for luciferase activity. As seen from FIGS. 2A and 2B, mice injected with AAV-AM showed a significantly higher luciferase signal than those injected with AAV-LK03.

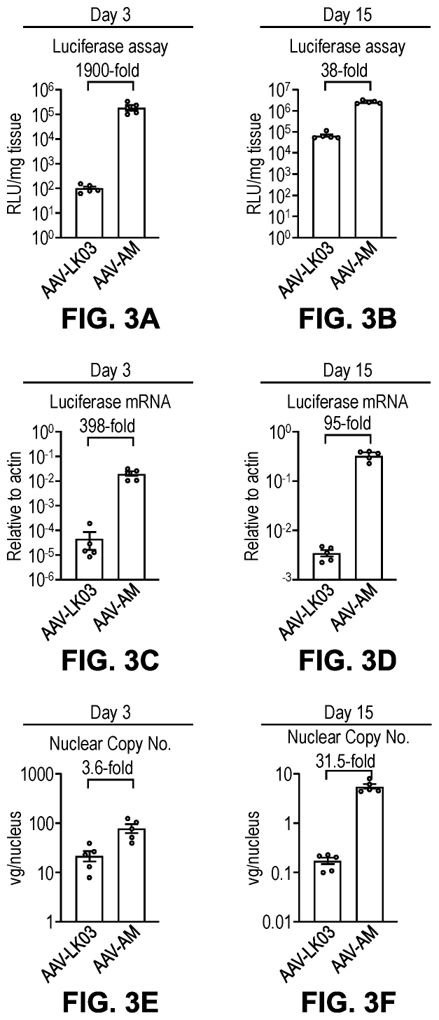

At day 3 and 15, animals in each group were sacrificed and liver tissues harvested for gene expression, protein activity and nuclear copy number analysis as described above. FIGS. 3A, 3C show a significantly higher expression of luciferase in the AAV-AM versus the AAV-LK03 group on day 3 post injection. Similar to the in vitro data, nuclear vector copy number was only modestly higher for the AAV-AM group (FIG. 3E). Importantly, copy number levels are different between AAV-LK03 and AAV-AAV-AM at Day 15, pointing out to a better preservation of the “gene of interest” transduced with the AAV-AM capsid.

While a number of exemplary aspects and embodiments have been illustrated and described those of skill in the art will recognize certain modifications, permutations, additions and sub-combinations thereof that can be made without departing from the spirit and scope of the invention(s). It is therefore intended that the following appended claims and claims hereafter introduced are interpreted to include all such modifications, permutations, additions and sub-combinations as are within their true spirit and scope, and it is to be understood that the disclosure is not to be limited to the specific embodiments disclosed herein, as such are presented by way of example. Although specific terms are employed herein, they are used in a generic and descriptive sense only and not for purposes of limitation.

All literature and similar materials cited in this application, including, but not limited to, patents, patent applications, articles, books, treatises, internet web pages and other publications cited in the present disclosure, regardless of the format of such literature and similar materials, are expressly incorporated by reference in their entirety for any purpose to the same extent as if each were individually indicated to be incorporated by reference. In the event that one or more of the incorporated literature and similar materials differs from or contradicts the present disclosure, including, but not limited to defined terms, term usage, described techniques, or the like, the present disclosure controls.

### Example 3

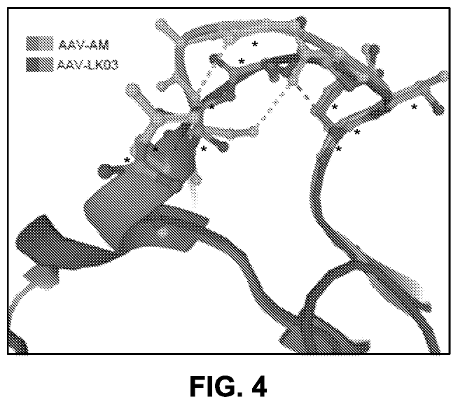

FIG. 4 shows a comparative predictive model for AAV-AM and AAV-LK03. Amino acid residues are marked with an asterisk. Non-covalent bonds are shown by the dashed lines in light grey and dark grey for AAV-AM and AAV-LK03, respectively.

TABLE 1
Amino acid and nucleic acid sequences
for rAAV capsid protein

| SEQ | | |
|---|---|---|
| ID | Descrip- | |
| NO | tion | Sequence |
| 1 | AAV-AM | MAADGYLPDWLEDNLSEGIREWWAL |
| | capsid | QPGAPKPKANQQHQDNARGLVLPGY |
| | protein | KYLGPGNGLDKGEPVNAADAAALEH |
| | sequence | DKAYDQQLKAGDNPYLKYNHADAEF |
| | | QERLKEDTSFGGNLGRAVFQAKKRL |
| | | LEPLGLVEEAAKTAPGKKRPVDQSP |
| | | QEPDSSSGVGKSGKQPARKRLNFGQ |
| | | TGDSESVPDPQPLGEPPAAPTSLGS |
| | | NTMASGGGAPMADNNEGADGVGNSS |
| | | GNWHCDSQWLGDRVITTSTRTWALP |
| | | TYNNHLYKQISSQSGGASNDNHYFG |
| | | YSTPWGYFDFNRFHCHFSPRDWQRL |
| | | INNNWGFRPKKLSFKLFNIQVKEVT |
| | | QNDGTTTIANNLTSTVQVFTDSEYQ |
| | | LPYVLGSAHQGCLPPFPADVFMVPQ |
| | | YGYLTLNNGSQAVGRSSFYCLEYFP |
| | | SQMLRTGNNFQFSYTFEDVPFHSSY |
| | | AHSQSLDRLMNPLIDQYLYYLNRTQ |
| | | GTTSGTTNQSRLLFSQAGPQSMSLQ |
| | | ARNWLPGPCYRQQRLSKTANDNNNS |
| | | NFPWTAASKYHLNGRDSLVNPGPAM |
| | | ASHKDDEEKFFPMHGNLIFGKEGTT |
| | | ASNAELDNVMITDEEEIRTTNPVAT |
| | | EQYGTVANNLQSSNTAPTTRTVNDQ |
| | | GALPGMVWQDRDVYLQGPIWAKIPH |
| | | TDGHFHPSPLMGGFGLKHPPPQIMI |
| | | KNTPVPANPPTTFSPAKFASFITQY |
| | | STGQVSVEIEWELQKENSKRWNPEI |
| | | QYTSNYNKSVNVDFTVDTNGVYSEP |
| | | RPIGTRYLTRPL |
| 2 | AAV-AM | ATGGCTGCTGACGGTTATCTTCCAG |
| | nucleotide | ATTGGCTCGAGGACAACCTTTCTGA |
| | sequence | AGGCATTCGAGAGTGGTGGGCGCTG |
| | | CAACCTGGAGCCCCTAAACCCAAGG |
| | | CAAATCAACAACATCAGGACAACGC |
| | | TCGGGGTCTTGTGCTTCCGGGTTAC |
| | | AAATACCTCGGACCCGGCAACGGAC |
| | | TCGACAAGGGGGAACCCGTCAACGC |
| | | AGCGGACGCGGCAGCCCTCGAGCAC |
| | | GACAAGGCCTACGACCAGCAGCTCA |
| | | AGGCCGGTGACAACCCCTACCTCAA |
| | | GTACAACCACGCCGACGCCGAGTTC |
| | | CAGGAGCGGCTCAAAGAAGATACGT |
| | | CTTTTGGGGGCAACCTCGGGCGAGC |
| | | AGTCTTCCAGGCCAAAAAGAGGCTT |
| | | CTTGAACCTCTTGGTCTGGTTGAGG |
| | | AAGCGGCTAAGACGGCTCCTGGAAA |
| | | GAAGAGGCCTGTAGATCAGTCTCCT |
| | | CAGGAACCGGACTCATCATCTGGTG |
| | | TTGGCAAATCGGGCAAACAGCCTGC |
| | | CAGAAAAAGACTAAATTTCGGTCAG |
| | | ACTGGCGACTCAGAGTCAGTCCCAG |
| | | ACCCTCAACCTCTCGGAGAACCACC |
| | | AGCAGCCCCCACAAGTTTGGGATCT |
| | | AATACAATGGCTTCAGGCGGTGGCG |
| | | CACCAATGGCAGACAATAACGAGGG |
| | | TGCCGATGGAGTGGGTAATTCCTCA |
| | | GGAAATTGGCATTGCGATTCCCAAT |
| | | GGCTGGGCGACAGAGTCATCACCAC |
| | | CAGCACCAGAACCTGGGCCCTGCCC |
| | | ACTTACAACAACCATCTCTACAAGC |
| | | AAATCTCCAGCCAATCAGGAggaGC |
| | | TTCAAACGACAACCACTACTTTGGC |
| | | TACAGCACCCCTTGGGGGTATTTTG |
| | | ACTTTAACAGATTCCACTGCCACTT |
| | | CTCACCACGTGACTGGCAGCGACTC |
| | | ATTAACAACAACTGGGGATTCCGGC |
| | | CCAAGAAACTCAGCTTCAAGCTCTT |
| | | CAACATCCAAGTTAAAGAGGTCACG |
| | | CAGAACGATGGCACGACGACTATTG |
| | | CCAATAACCTTACCAGCACGGTTCA |
| | | AGTGTTTACGGACTCGGAGTATCAG |
| | | CTCCCGTACGTGCTCGGGTCGGCGC |
| | | ACCAAGGCTGTCTCCCGCCGTTTCC |
| | | AGCGGACGTCTTCATGGTCCCTCAG |
| | | TATGGATACCTCACCCTGAACAACG |
| | | GAAGTCAAGCGGTGGGACGCTCATC |
| | | CTTTTACTGCCTGGAGTACTTCCCT |
| | | TCGCAGATGCTAAGGACTGGAAATA |
| | | ACTTCCAATTCAGCTATACCTTCGA |
| | | GGATGTACCTTTTCACAGCAGCTAC |
| | | GCTCACAGCCAGAGTTTGGATCGCT |
| | | TGATGAATCCTCTTATTGATCAGTA |
| | | TCTGTACTACCTGAACAGAACGCAA |
| | | GGAACAACCTCTGGAACAACCAACC |
| | | AATCACGGCTGCTTTTTAGCCAGGC |
| | | TGGGCCTCAGTCTATGTCTTTGCAG |
| | | GCCAGAAATTGGCTACCTGGGCCCT |
| | | GCTACCGGCAACAGAGACTTTCAAA |
| | | GACTGCTAACGACAACAACAACAGT |
| | | AACTTTCCTTGGACAGCGGCCAGCA |
| | | AATATCATCTCAATGGCCGCGACTC |
| | | GCTGGTGAATCCAGGACCAGCTATG |
| | | GCCAGTCACAAGGACGATGAAGAAA |
| | | AATTTTTCCCTATGCACGGCAATCT |
| | | AATATTTGGCAAAGAAGGGACAACG |
| | | GCAAGTAACGCAGAATTAGATAATG |
| | | TAATGATTACGGATGAAGAAGAGAT |
| | | TCGTACCACCAATCCTGTGGCAACA |
| | | GAGCAGTATGGAACTGTGGCAAATA |
| | | ACTTGCAGAGCTCAAATACAGCTCC |
| | | CACGACTAGAACTGTCAATGATCAG |
| | | GGGGCCTTACCTGGCATGGTGTGGC |
| | | AAGATCGTGACGTGTACCTTCAAGG |
| | | ACCTATCTGGGCAAAGATTCCTCAC |
| | | ACGGATGGACACTTTCATCCTTCTC |
| | | CTCTGATGGGAGGCTTTGGACTGAA |
| | | ACATCCGCCTCCTCAAATCATGATC |
| | | AAAAATACTCCGGTACCGGCAAATC |
| | | CTCCGACGACTTTCAGCCCGGCCAA |
| | | GTTTGCTTCATTTATCACTCAGTAC |
| | | TCCACTGGACAGGTCAGCGTGGAAA |
| | | TTGAGTGGGAGCTACAGAAAGAAAA |
| | | CAGCAAACGTTGGAATCCAGAGATT |
| | | CAGTACACTTCCAACTACAACAAGT |
| | | CTGTTAATGTGGACTTTACTGTAGA |
| | | CACTAATGGTGTTTATAGTGAACCT |
| | | CGCCCCATTGGCACCCGTTACCTTA |
| | | CCCGTCCCCTGTAA |
| 3 | AAV-LK03 | MAADGYLPDWLEDNLSEGIREWWAL |
| | capsid | QPGAPKPKANQQHQDNARGLVLPGY |
| | protein | KYLGPGNGLDKGEPVNAADAAALEH |
| | sequence | DKAYDQQLKAGDNPYLKYNHADAEF |
| | | QERLKEDTSFGGNLGRAVFQAKKRL |
| | | LEPLGLVEEAAKTAPGKKRPVDQSP |
| | | QEPDSSSGVGKSGKQPARKRLNFGQ |
| | | TGDSESVPDPQPLGEPPAAPTSLGS |
| | | NTMASGGGAPMADNNEGADGVGNSS |
| | | GNWHCDSQWLGDRVITTSTRTWALP |
| | | TYNNHLYKQISSQSGASNDNHYFGY |
| | | STPWGYFDFNRFHCHFSPRDWQRLI |
| | | NNNWGFRPKKLSFKLFNIQVKEVTQ |
| | | NDGTTTIANNLTSTVQVFTDSEYQL |
| | | PYVLGSAHQGCLPPFPADVFMVPQY |
| | | GYLTLNNGSQAVGRSSFYCLEYFPS |
| | | QMLRTGNNFQFSYTFEDVPFHSSYA |
| | | HSQSLDRLMNPLIDQYLYYLNRTQG |
| | | TTSGTTNQSRLLFSQAGPQSMSLQA |
| | | RNWLPGPCYRQQRLSKTANDNNNSN |
| | | FPWTAASKYHLNGRDSLVNPGPAMA |
| | | SHKDDEEKFFPMHGNLIFGKEGTTA |
| | | SNAELDNVMITDEEEIRTTNPVATE |
| | | QYGTVANNLQSSNTAPTTRTVNDQG |
| | | ALPGMVWQDRDVYLQGPIWAKIPHT |
| | | DGHFHPSPLMGGFGLKHPPPQIMIK |
| | | NTPVPANPPTTFSPAKFASFITQYS |
| | | TGQVSVEIEWELQKENSKRWNPEIQ |
| | | YTSNYNKSVNVDFTVDTNGVYSEPR |
| | | PIGTRYLTRPL |

1. A capsid protein sequence, comprising: an amino acid sequence of SEQ ID NO: 1 (AAV-AM).

2. A nucleotide sequence encoding a recombinant capsid protein, wherein the nucleotide sequence is SEQ ID NO: 2, or a sequence having at least about 85% sequence identity, at least about 90% sequence identity, at least about 95% sequence identity, at least about 96% sequence identity, at least about 97% sequence identity, at least about 98% sequence identity, or at least about 99% sequence identity thereto.

3. An amino acid sequence encoded by a nucleotide sequence of claim 2.

4. A viral particle, comprising the sequence of claim 1.

5. A viral particle, comprising: an amino acid sequence for a recombinant capsid protein encoded by a nucleotide sequence of claim 2.

6. A plasmid, comprising: a nucleotide encoding the capsid protein sequence of claim 1.

7. A plasmid, comprising a nucleotide sequence for a recombinant capsid protein encoded by the nucleotide sequence of claim 2.

8. A recombinant AAV vector, comprising: the capsid protein sequence of claim 1.

9. A recombinant AAV vector, comprising: an amino acid sequence for a capsid protein, said amino acid sequence encoded by the nucleotide sequence of claim 3.

10. A method of transferring a nucleic acid sequence of interest into a cell or into a mammal, comprising:
introducing a recombinant AAV (rAAV) vector into a cell or a mammal, the rAAV vector encoding a recombinant capsid nucleotide sequence and a gene of interest, wherein the gene of interest is encapsidated into the recombinant capsid protein having the capsid protein sequence of claim 1.

11. A method of transferring a nucleic acid sequence of interest into a cell or into a mammal, comprising:
introducing a recombinant AAV (rAAV) vector into a cell or a mammal, the recombinant AAV vector encoding a recombinant capsid nucleotide sequence and a gene of interest, wherein the gene of interest is encapsidated into a recombinant capsid protein having an amino acid encoded by a nucleotide sequence of claim 2.

12. The method of claim 10, wherein the recombinant AAV vector is generated by steps comprising:
(a) performing a site directed mutagenesis of an AAV-LK03 plasmid using overlapping primers comprising an extraneous triplet nucleotide sequence;
(b) performing an exponential amplification by PCR using the AAV-LK03 plasmid as template and primer pairs comprising the extraneous triplet nucleotide sequence to generate amplicons;
(c) incubating the amplicons with a kinase, Ligase and DpnII;
(d) performing a high efficiency transformation in bacteria;
(e) culturing the bacteria in the presence of a selection antibiotic to isolate positive bacterial clones comprising a recombinant capsid nucleic acid sequence;
(f) confirming by sequencing the presence of the recombinant capsid nucleotide sequence; and
(g) purifying plasmids comprising the recombinant capsid nucleic acid sequence from the positive bacterial clones; thereby generating the recombinant AAV vector.

13. The method of claim 10, wherein the capsid nucleotide sequence codes for a capsid protein sequence comprising an extraneous amino acid.

14. The method of claim 12, wherein the extraneous amino acid is inserted at position 266 in the capsid protein sequence.

15. The method of claim 13, wherein the extraneous amino acid is glycine.

16. The method of claim 11, wherein the cell is a HuH-7 cell, a Hepa1-6 cell, a HEK293 cell, or a hPAEC cell.

17. The method of claim 11, wherein the mammal is a rodent or a human.

18. The method of claim 17, wherein the rodent is a mouse.

19. The method of claim 11, wherein the rAAV is introduced into the liver tissue or skin of the mammal.

20. A method of determining the therapeutic efficacy for a gene of interest comprising:
delivering to a first mammal species, a recombinant AAV (rAAV) vector comprising a nucleotide sequence encoding a recombinant capsid protein with an amino acid sequence as set forth in SEQ ID NO: 1 (AAV-AM), and a gene of interest, wherein the gene of interest is encapsidated into the recombinant capsid protein;
assessing an efficiency of transduction in the first mammal;
delivering to a second mammal species, the rAAV vector when the efficiency of transduction in the first mammal species is higher than that observed after infecting a first mammal species with a control rAAV vector;
assessing an efficiency of transduction in the second mammal species, whereby, a higher efficiency of transduction of the rAAV vector in the first mammal species and the second mammal species over the control rAAV vector is indicative of a higher therapeutic efficacy for the gene of interest.

21. The method of claim 20, wherein the first mammal species is a rodent.

22. The method of claim 20, wherein the second mammal species is a human.

23. The method of claim 20, wherein the first mammal species is a mouse, and the second mammal species is a human.

24. The method of claim 20, wherein the rAAV vector comprising the gene of interest is transduced in the liver.

25. The method of claim 20, wherein the control rAAV vector comprises the AAV-LK03 plasmid.

26. The method of claim 20, wherein the efficiency of transduction is determined by measuring at least one of, a nuclear vector copy number, expression of the gene of interest, and activity of a protein encoded by the gene of interest.

27. The nucleotide sequence of claim 2, wherein said nucleotide sequence has at least about 96% or at least about 97% or at least about 98% or at least about 99% sequence identity to SEQ ID NO: 2.

28. (canceled)

29. A nucleotide sequence as in claim 27, wherein said nucleotide sequence encodes an amino acid sequence that is not identical to SEQ ID NO: 3.

30. The nucleotide sequence of claim 27, wherein said nucleotide sequence comprises SEQ ID NO: 2.

31. The nucleotide sequence of claim 27 recombinant capsid protein, wherein said nucleotide sequence consists of SEQ ID NO: 2.

32. A plasmid, comprising a nucleotide sequence as in claim 27.

33. A plasmid, comprising a nucleotide sequence as in claim 27, wherein said nucleotide sequence encodes an amino acid sequence that is not identical to SEQ ID NO: 3.

34. A recombinant AAV vector, comprising: a nucleotide sequence as in claim 27.

35. A recombinant AAV vector, comprising: a nucleotide sequence as in claim 27, wherein said nucleotide sequence encodes an amino acid sequence that is not identical to SEQ ID NO: 3.


SEQUENCE LISTING (3 sequences)

SEQ ID NO: 1 - protein, 737 aa, synthetic construct
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 2 - DNA, 2214 bp, synthetic construct
  source 1..2214: mol_type = other DNA
atggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaa

SEQ ID NO: 3 - protein, 736 aa, synthetic construct
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL